(19) United States
(12) Patent Application Publication
(10) Pub. No.: US 2023/0414963 A1
(43) Pub. Date: Dec. 28, 2023

(54) MULTI-MODAL SKIN TREATMENT DEVICE

(71) Applicant: Skin Design, LLC, Santa Monica, CA (US)

(72) Inventors: Ava T. SHAMBAN, Los Angeles, CA (US); Rich CAMPBELL, Los Angeles, CA (US); Emily GASTA, Watertown, MA (US)

(73) Assignee: Skin Design, LLC, Santa Monica, CA (US)

(21) Appl. No.: 17/664,128

(22) Filed: May 19, 2022

(51) Int. Cl. A61N 5/06; A61N 1/40; A61N 1/36

(52) U.S. Cl. CPC A61N 5/0616; A61N 1/40; A61N 1/36003; A61N 2005/0626; A61N 2005/0652; A61N 2005/0663; A61N 1/36034

(57) ABSTRACT

A device for skin treatment includes an LED (light-emitting diode) system that is configured to emit light at a first wavelength, a first radiofrequency (RF) system that is configured to generate a first electrical signal, and a second RF system that is configured to generate a second electrical signal. The first RF system, the second RF system, and the LED system are configurable to operate in a first treatment mode, causing the device to provide the first electrical signal, the second electrical signal, and light at the first wavelength.

### TECHNICAL FIELD

This disclosure relates to skin treatment techniques, and particularly to treatment techniques using radiofrequency energy, electrical energy, light, and other modalities.

### BACKGROUND

Electro-muscular stimulation (EMS) utilizes microcurrent electrical impulses to stimulate nerves. When an EMS device is used on a treatment area, the muscle groups in the treatment area expand and contract. The result is a toned look, and the skin gets tighter and smoother.

Radiofrequency (RF) therapy uses low energy radiation to heat the deep layer of the skin called the dermis. This heat stimulates the production of collagen to help improve signs of wrinkles and sagging skin. Research has found that RF therapy is usually safe and can be effective at treating mild or moderate signs of aging.

Light-emitting diode (LED) light therapy is used to accelerate wound healing and to help regenerate damaged muscle tissues. LED light therapy is also used in aesthetics, for example, to increase collagen in tissues. All of which can smooth out the skin and reduce the appearance of damage from age spots, acne, wrinkles.

In electroporation treatments, short high-voltage pulses are used to overcome the barrier of the cell membrane. By applying an external electric field, which surpasses the capacitance of the cell membrane, transient and reversible breakdown of the membrane can be induced.

### SUMMARY

Embodiments of the disclosure include a device for skin treatment. The device may include an LED (light-emitting diode) system that is configured to emit light at a first wavelength; a first radiofrequency (RF) system that is configured to generate a first electrical signal; and a second RF system that is configured to generate a second electrical signal, where the device is configurable to operate in a first treatment mode, where the first RF system, the second RF system, and the LED system are configurable to operate in the first treatment mode, causing the device to provide the first electrical signal, the second electrical signal, and light at the first wavelength.

Embodiments of the disclosure provide methods for skin treatment. The method includes receiving a first user input to select a first treatment mode; and in response to selecting the first treatment mode, providing light at a first wavelength from an LED system, a first electrical signal from a first RF (radiofrequency) system, and a second electrical signal from a second RF system.

### BRIEF DESCRIPTION OF THE DRAWINGS

Further objectives and advantages will become apparent from a consideration of the description, drawings, and examples.

FIG. 1 is a diagram illustrating a multi-modal skin treatment device according to various embodiments.

FIG. 2A shows a front perspective view of a multi-modal skin treatment device according to some embodiments.

FIG. 2B shows a rear view of a multi-modal skin treatment device according to various embodiments.

FIG. 2C shows a detail view of an LED array in a multi-modal skin treatment device according to various embodiments.

FIG. 3 shows a cross section view, taken along line 3-3 in FIG. 2A, of the multi-modal skin treatment device according to various embodiments.

FIG. 4A shows a process for selecting treatment mode performed by a multi-modal skin treatment device of some embodiments.

FIG. 4B shows a process for selecting a power setting performed by a multi-modal skin treatment device of some embodiments.

FIG. 4C shows a process for selecting a treatment duration performed by a multi-modal skin treatment device of some embodiments.

FIG. 5 shows a sequence of user interactions with a user interface to select the treatment mode, power setting, and treatment duration for a multi-modal skin treatment device of some embodiments.

FIG. 6 conceptually illustrates an electronic system with which some embodiments of the invention are implemented.

### DETAILED DESCRIPTION

Some embodiments of the invention are discussed in detail below. In describing embodiments, specific terminology is employed for the sake of clarity. However, the invention is not intended to be limited to the specific terminology so selected. A person skilled in the relevant art will recognize that other equivalent components can be employed, and other methods developed, without departing from the broad concepts of the current invention. All references cited anywhere in this specification, including the Background and Detailed Description sections, are incorporated by reference as if each had been individually incorporated.

Some embodiments provide a multi-modal device for skin treatment. Specifically, some embodiments of the device include RF systems and LED systems that may be configured to provide separately or in combination any one or more of electro-muscular stimulation (EMS) therapy, radiofrequency (RF) therapy, light-emitting diode (LED) therapy, and electroporation therapy to a targeted area of the skin.

In some embodiments, the device includes an LED (light-emitting diode) system that is configured to emit light at a first wavelength, a first radiofrequency (RF) system that is configured to generate a first electrical signal, and a second RF system that is configured to generate a second electrical signal. The device is configurable to operate in a first treatment mode. The first RF system, the second RF system, and the LED system are configurable to operate in the first treatment mode, causing the device to provide light at the first wavelength, the first electrical signal, and the second electrical signal. The first electrical signal may be configured to provide therapy, including but not limited to RF therapy or EMS therapy. The second electrical signal may be configured to provide therapy, including but not limited to RF therapy or EMS therapy. The first wavelength may be a wavelength that includes but is not limited to a blue wavelength and a red wavelength.

In some embodiments, the device is configurable to operate in a second treatment mode, and the LED system is further configured to emit light at a second wavelength. The first RF system, the second RF system, and the LED system are configurable to operate in the second treatment mode, causing the device to provide the first electrical signal, the second electrical signal, and light at the second wavelength. The first electrical signal may be configured to provide therapy, including but not limited to RF therapy or EMS therapy. The second electrical signal may be configured to provide therapy, including but not limited to RF therapy or EMS therapy. The second wavelength is a different wavelength than the first wavelength, and may be a wavelength that includes but is not limited to a blue wavelength and a red wavelength.

In some embodiments, the device is configurable to operate in a third treatment mode. The first RF system, the second RF system, and the LED system are configurable to operate in the third treatment mode, causing the device to provide the first electrical signal, the second electrical signal, light at the first wavelength, and light at the second wavelength. The first electrical signal may be configured to provide therapy, including but not limited to RF therapy or EMS therapy. The second electrical signal may be configured to provide therapy, including but not limited to RF therapy or EMS therapy. The first wavelength may be a wavelength that includes but is not limited to a blue wavelength and a red wavelength. The second wavelength is a different wavelength than the first wavelength, and may be a wavelength that includes but is not limited to a blue wavelength and a red wavelength.

In some embodiments, one or more of the treatment modes may include providing one or more of the first electrical signal, second electrical signal, and light at various wavelengths simultaneously, substantially simultaneously, or at intervals. The first electrical signal may be configured to provide therapy, including but not limited to RF therapy or EMS therapy. The second electrical signal may be configured to provide therapy, including but not limited to RF therapy or EMS therapy. The various wavelengths may include but are not limited to a blue wavelength and a red wavelength.

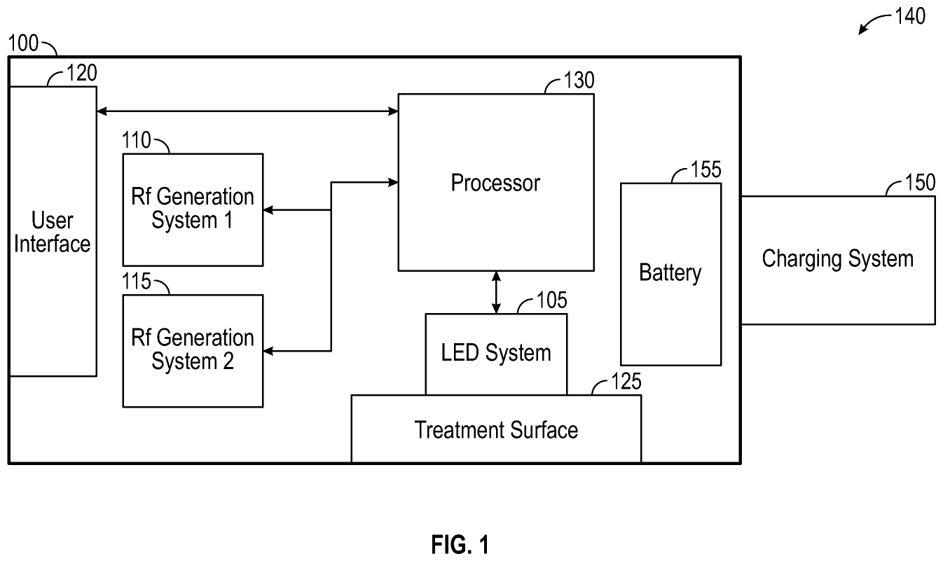

FIG. 1 is a diagram illustrating a multi-modal skin treatment device according to various embodiments. In the example shown, a multi-modal skin treatment device 100 includes an LED system 105, a first RF generation system 110, and a second RF generation system 115. The device 100 also includes a user interface 120 and a treatment surface 125. A processor 130 is communicatively coupled to the user interface 120, the LED system 105, and the RF generation systems 110, 115.

During operation of the device 100, a user configures the device 100 using the user interface 120 and positions the device 100 so that the treatment surface 125 is in contact with the user's skin. The LED system 105 is coupled to the treatment surface 125 so that light generated by the LED system 105 is directly emitted from the treatment surface 125 onto the user's skin. The RF generation systems 110, 115 are electrically coupled to the treatment surface 125 so that electrical signals generated by the RF generation systems 110, 115 are provided from the treatment surface 125. In some embodiments, the RF generation systems 110, 115 are coupled to electrodes (not shown in FIG. 1) on the treatment surface, such that the electrodes provide the electrical signals generated by the RF generation systems 110, 115 to the user's skin.

In some embodiments, the device 100 is part of a treatment system 140 that also includes a device charging system 150. The device charging system 150 may physically couple to the device 100 in order to provide electrical power to the device 100. The device 100 may have an internal battery 155 which receives power from the charging system 150 when the charging system 150 is physically coupled to the device 100. The internal battery 155 then provides power to the components of the device 100, including but not limited to the processor 130, the user interface 120, the LED system 105, and the RF generation systems 110, 115. For example, the internal battery 155 may provide power to the components of the device 100 via a power bus (not shown in FIG. 1).

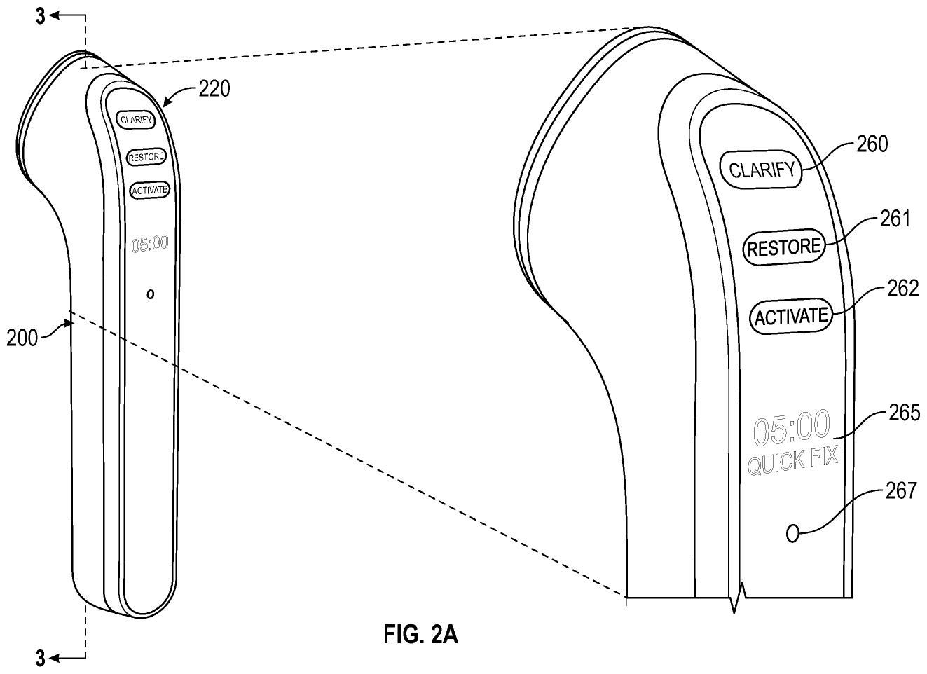

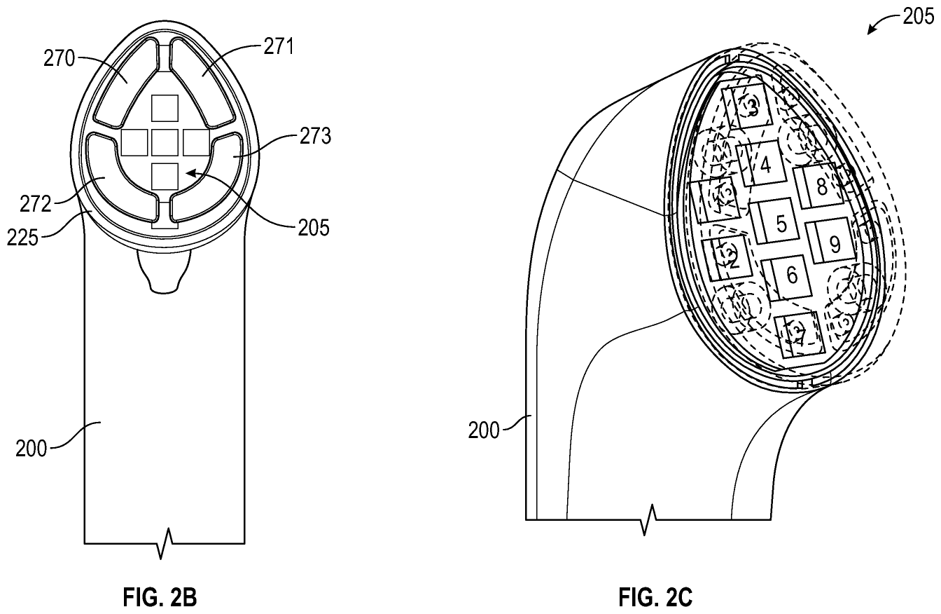

FIG. 2A shows a front perspective view of a multi-modal skin treatment device according to some embodiments. In the example shown, a multi-modal skin treatment device 200 is similar to the embodiment of the device 100 discussed above with respect to FIG. 1, and like reference numerals have been used to refer to the same or similar components. A detailed description of these components will be omitted, and the following discussion focuses on the differences between these embodiments. Any of the various features discussed with any one of the embodiments discussed herein may also apply to and be used with any other embodiments.

A user interface 220 is shown in the perspective view of FIG. 2A, and magnified in the detail view on the right. The device 200 can be configured to operate in multiple treatment modes (e.g., CLARIFY, RESTORE, and ACTIVATE treatment modes) by pressing one of the corresponding labeled buttons 260, 261, 262 on the user interface 220. The user interface 220 also includes a display 265, which indicates the remaining treatment time once one of the treatment modes is selected using buttons 260-262 and the device 200 begins operation. The display 265 may also display additional information, such as what power setting the device 200 is currently in. In addition, the user interface 220 includes a battery charge indicator light 267, which may change color or brightness, as well as pulse or flash in a pattern, to indicate various states such as charging, ready, battery full, battery low, emitting energy for treatment, treatment almost complete, etc.

In some embodiments, the device further includes a first electrode pair operatively connected to the first RF system, and a second electrode pair operatively connected to the second RF system. The LED system, the first electrode pair, and the second electrode pair are mounted on a treatment surface at one end of the device. In some such embodiments, the treatment surface has a teardrop shape. Though, other shapes may be used depending on, for example, the intended treatment area.

FIG. 2B shows a rear view of a multi-modal skin treatment device according to various embodiments. In the example shown, a multi-modal skin treatment device 200 includes a rear view of the treatment device shown in FIG. 2A. The treatment surface 225 is visible in the rear view of FIG. 2B, and is covered by a clear base (e.g., formed of a transparent or translucent plastic material), with four electrodes 270, 271, 272, 273 mounted above the clear base. In this example, the treatment surface 225 is teardrop shaped, to allow the treatment surface to be positioned by the user on the user's skin as desired. In other embodiments, the treatment surface 225 may be a different shape, including but not limited to oval, circular, diamond, or other shape. In certain cases, a treatment surface 225 may be larger or smaller, to facilitate different sizes of treatment areas on the skin. Smaller treatment surfaces are suitable for treating smaller regions such as the face, and larger treatment surfaces are suitable for treating larger regions such as the skin on the torso and the limbs.

The electrodes 270-273 include conductive surfaces grouped into diagonal pairs. A first pair of electrodes 270, 273 serve as an anode-cathode pair for an electrical signal generated by an RF generation system, for example, inside the device 200. A second pair of electrodes 271, 272 serve as an anode-cathode pair for an electrical signal generated by another RF generation system, for example, inside the device 200. In certain cases, the first pair of electrodes 270, 273 may provide RF therapy treatment and the second pair of electrodes 271, 272 provide EMS therapy treatment. In certain cases, an RF therapy treatment may include application of RF energy at higher relative voltages and EMS therapy treatment may include application of RF energy at lower relative voltages. The electrode pairs are placed diagonally in this example, so that they contact the skin more evenly. In some embodiments, the electrodes 270-273 are raised slightly above the treatment surface 225, to optimize the performance of the treatments. In the example of FIG. 2B, the electrodes 270-273 are raised 1 millimeter, and have rounded edges to facilitate easier cleaning. However, other configurations are contemplated within the scope of this disclosure.

In the example of FIG. 2B, there are only two electrode pairs, though in other embodiments there may be fewer or more electrode pairs. For example, in some embodiments, at least one RF generator may be connected to more than one electrode pair, to provide a larger treatment area for the electrical signal generated by that generator. In some embodiments, there may be more than two RF generators, each connected to at least one electrode pair. As an example, some embodiments may have a large treatment surface with four electrode pairs, with two RF generators connected to two electrode pairs each.

FIG. 2C shows a detail view of an LED array in a multi-modal skin treatment device according to various embodiments. In the example shown, an LED array 205 is included in proximity to the treatment surface 225, for example, located below the clear plastic base and the electrodes 270-273. FIG. 2C shows a detail view of the LED array 205, which in this example has nine separate LED sources. In other embodiments, there may be a different number of LED sources in the LED array 205, arranged in any suitable pattern in the treatment surface 225. The LED sources generate light that passes through the clear plastic base and is partly scattered thereby, before shining upon the skin. In some embodiments, the clear plastic base is frosted (semi-transparent), to increase the amount of scattering for a more even irradiation of the skin by the LED sources. The LED sources may be arranged in a pattern to optimize the effective treatment area. In certain cases, LED array 205 may include a maximum number of LED sources possible given the space available in the treatment surface 225. The LED array 205 may include LED sources arranged in a pattern that is, for example, optimized for certain treatments, treatment areas, or other factors.

In some embodiments, the LED sources may be positioned relative to the electrodes 270-273 to increase the effectiveness of a combined LED and RF treatment. For example, the LED sources may be positioned so that LED energy and RF energy are focused on overlapping treatment areas in the skin, for example, to maximize the treatment effect.

In various embodiments, LED sources can shine at a red wavelength, a blue wavelength, or other wavelengths, depending on the treatment mode. In the example of FIG. 2C, the LED sources include a dual-LED package capable of emitting light at two different primary wavelengths. When the device 200 is set to one treatment mode (e.g., a CLARIFY treatment mode) all nine LED sources emit blue light, e.g., light with a peak wavelength between 405-420 nanometers. When the device 200 is set to another treatment mode (e.g., a RESTORE treatment mode) all nine LED sources emit red light, e.g., light with a peak wavelength between 630-660 nanometers. When the device 200 is set to a further treatment mode (e.g., the ACTIVATE treatment mode) all nine LED sources emit both red and blue light, e.g., light with two peak wavelengths, one peak wavelength between 405-420 nanometers and the other peak wavelength between 630-660 nanometers. In other embodiments, the LED sources may emit light in other ranges of wavelengths, including but not limited to wavelengths of visible light, infrared light, or ultraviolet light.

In some embodiments, LED sources may include multiple single-color LEDs, each capable of emitting light at a single primary wavelength. The LED sources may emit light in various combinations. For example, a first subset of these single-color LEDs may be configured to emit blue light, and a second subset may be configured to emit red light. In other embodiments, the single-color LEDs may emit light at other wavelengths, including but not limited to wavelengths of visible light, infrared light, or ultraviolet light. Accordingly, during different treatment modes, one or both of the subsets of single-color LEDs may be active.

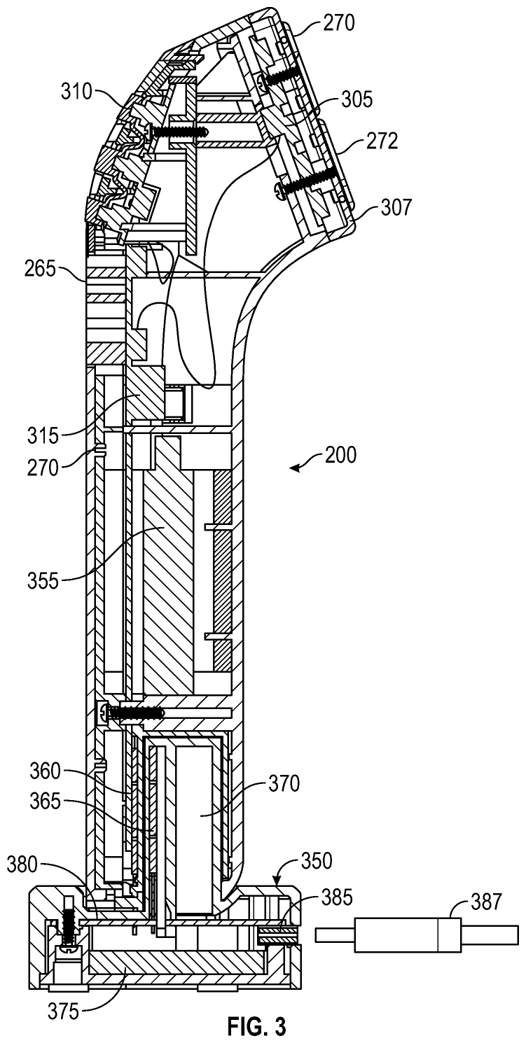

FIG. 3 shows a cross section view, taken along line 3-3 in FIG. 2A, of the multi-modal skin treatment device 200 according to various embodiments. In this view, several printed circuit board assemblies (PCBAs) are shown. For example, the device 200 has a treatment PCBA 305, which houses the LED array 205 and the RF generators. The treatment PCBA 305 may include a frosted or clear plastic cover 307 to permit light from the LED array 205 to pass through and to diffuse that light onto the user's skin. The treatment electrodes 270-273 are mounted above the cover 307 so as to come into direct contact with the user's skin.

The device 200 also has a user interface PCBA 310, which houses the treatment mode selection buttons 260-262, the display 265, or the indicator light 267. The device further includes a main PCBA 315, which houses a processor (e.g., processor 130 of FIG. 1) that receives input from the user interface PCBA 310 and sends commands to the treatment PCBA 305. The processor 130 may also receive feedback from the treatment PCBA 305 and send configuration information to the user interface PCBA 310, for example, to set the color or flashing of the indicator light 267, to illuminate or darken one or more of the buttons 260-262, and to control what is displayed on the display 265. The main PCBA 315 may also include a power bus, which receives power from an external charging stand 350, charges an internal battery 355, and provides power from the internal battery 355 to the treatment PCBA 305 and the user interface PCBA 310. The main PCBA 315 may receive the power from the charging stand 350 using an induction charging coil 360. In some embodiments, the functions of the treatment PCBA 305, the user interface PCBA 310, and the main PCBA 315 may be performed by fewer PCBAs or more PCBAs, for example, by a single PCBA, separate PCBAs for each treatment modality, and the like.

The charging stand 350 also has a charging induction coil 365, which wirelessly couples to the device induction charging coil 360, and is positioned in a vertical flange 370. The device induction charging coil 360 is aligned in proximity to the charging stand charging induction coil 365 when the device 200 is positioned onto the charging stand 350. The device 200 has a slot to receive the vertical flange 370 of the charging stand 350, and the charging stand 350 also has a weight 375 to provide stability when the device 200 is docked and charging. The charging stand 350 also includes a charging PCBA 380 to regulate the power provided to the device 200, and a port 385 (e.g., a USB-C port) that receives a power plug 387.

In some embodiments, the device is configurable by a user to operate at one of a low power setting and a high power setting. In the high power setting, one or more of the voltage, duty cycle, and duty ratio may be higher relative to the low power setting.

In some embodiments, the device is configurable by a user to operate at treatment modes of varying duration. For example, the device may operate at one of a long treatment mode and a short treatment mode. In certain cases, the device operates for ten minutes in the long treatment mode, and the device operates for three minutes in the short treatment mode. In some embodiments, the duration may range from one minute to thirty minutes and may preferably range from three minutes to fifteen minutes, and more preferably may range from three minutes to ten minutes.

In some embodiments, the first electrical signal may be configured to provide radiofrequency (RF) therapy. In some such embodiments, the frequency of the first electrical signal may range from 50 kHz to 100 kHz, and may preferably range from 75 kHz to 85 kHz. In some such embodiments, the duty cycle of the first electrical signal may range from 5 to 15 microseconds, and may preferably range from 12 microseconds to 12.2 microseconds. In some such embodiments, the duty ratio of the first electrical may range between 30% and 55% and may preferably range between 37.6% and 48%. In some such embodiments, the voltage of the first electrical signal may range between 60 to 90 volts, and may preferably range between 70 and 80 volts.

In some embodiments, the second electrical signal may be configured to provide electro-muscular stimulation (EMS) therapy. In some such embodiments, the frequency of the second electrical signal may range from 7 Hz to 10 Hz and may preferably range from 8.34 Hz to 8.65 Hz. In some such embodiments, the cycle of the second electrical signal may range from 95 milliseconds to 200 milliseconds and may preferably range from 115 milliseconds to 119 milliseconds. In some such embodiments, the duty cycle of the second electrical signal may range between 40% and 60%, and may preferably be 50%. In some such embodiments, the voltage of the second electrical signal may range between 5 to 25 volts, and may preferably range between 10.45 and 20.75 volts.

Table 1 shows different operating parameters for different treatment modes of the device 200 in some embodiments. In some embodiments, the operating parameters may be within a range of ±10% of the minimum and maximum values shown in Table 1.

TABLE 1

| Treatment | LED | Power | Treatment | | | |
|---|---|---|---|---|---|---|
| Mode | Colors | Mode | Duration | | RF Therapy | EMS Therapy |
| CLARIFY | Blue wavelength | Low | 10 | min | Freq: 81.77-82.96 KHZ | Freq: 8.46-8.50 HZ |
| | 412-415 nm | | | | Cycle: 12-12.2 us | Cycle: 117-118 ms |
| | Brightness | | | | Voltage: 70-78 V | Voltage: 10.45-11.45 v |
| | 0.22290-0.29326 lm | | | | Duty Ratio: 37.6-39% | Duty Ratio: 50% |
| | | High | 10 | min | Freq: 81.69-82.86 KHZ | Freq: 8.34-8.65 HZ |
| | | Quick Fix | 3 | min | Cycle: 12-12.2 us | Cycle: 115-119 ms |
| | | | | | Voltage: 72-76 V | Voltage: 13-14.65 v |
| | | | | | Duty Ratio: 45.6-47.6% | Duty Ratio: 50% |
| RESTORE | Red wavelength | Low | 10 | min | Freq: 81.70-82.96 KHZ | Freq: 8.47-8.58 HZ |
| | 656-657 nm | | | | Cycle: 12-12.2 us | Cycle: 116-118 ms |
| | Brightness | | | | Voltage: 70-76 V | Voltage: 16.9-17.5 V |
| | 0.55954-0.73041 lm | | | | Duty Ratio: 37.7-41% | Duty Ratio: 50% |
| | | High | 10 | min | Freq: 81.71-83.11 KHZ | Freq: 8.44-8.58 HZ |
| | | Quick Fix | 3 | min | Cycle: 12-12.2 us | Cycle: 116-118 ms |
| | | | | | Voltage: 72-76 V | Voltage: 20.1-20.75 V |
| | | | | | Duty Ratio: 45-47.6% | Duty Ratio: 50% |
| ACTIVATE | Blue wavelength | Low | 10 | min | Freq: 81.62-83.06 KHZ | Freq: 8.42-8.57 HZ |
| | 412-416 nm | | | | Cycle: 12-12.2 us | Cycle: 116-118 ms |
| | Red wavelength | | | | Voltage: 70-76 V | Voltage: 16.9-17.4 V |
| | 656-657 nm | | | | Duty Ratio: 35.5-40% | Duty Ratio: 50% |
| | Brightness | High | 10 | min | Freq: 81.86-82.91 KHZ | Freq: 8.35-8.60 HZ |
| | 0.70747-0.93618 lm | Quick Fix | 3 | min | Cycle: 12-12.2 us | Cycle: 116-119 ms |
| | | | | | Voltage: 72-76 V | Voltage: 20.1-20.75 V |
| | | | | | Duty Ratio: 45.6-48% | Duty Ratio: 50% |

In one example treatment mode, e.g., the CLARIFY treatment mode, the device 200 provides high-voltage RF therapy from the first pair of electrodes 270, 273, and micro-voltage EMS therapy through the second pair of electrodes 271, 272, while also providing blue light from the LED array 205.

In another example treatment mode, e.g., the RESTORE treatment mode, the device 200 provides high-voltage RF therapy from the first pair of electrodes 270, 273, and low-voltage EMS therapy through the second pair of electrodes 271, 272, while also providing red light from the LED array 205.

In a further example treatment mode, e.g., the ACTIVATE treatment mode, the device 200 provides high-voltage RF therapy from the first pair of electrodes 270, 273, and low-voltage EMS therapy through the second pair of electrodes 271, 272, while also providing a combination of blue and red light from the LED array 205.

In various treatment modes, the device 200 may be operated at a low power setting, a high power setting, or a quick fix setting. For example, in some embodiments, the low power setting is characterized by a baseline voltage, and the high power setting is characterized by a higher voltage than the baseline voltage. In some embodiments, the duration of the treatment in both the low power setting and the high power setting may be ten minutes, and in the quick fix setting, the voltage is set equal to the high power setting, but the duration is limited to three minutes. Specific voltage levels and other operating parameters used in some embodiments for each treatment mode, and each power setting within each treatment mode, are summarized in Table 1.

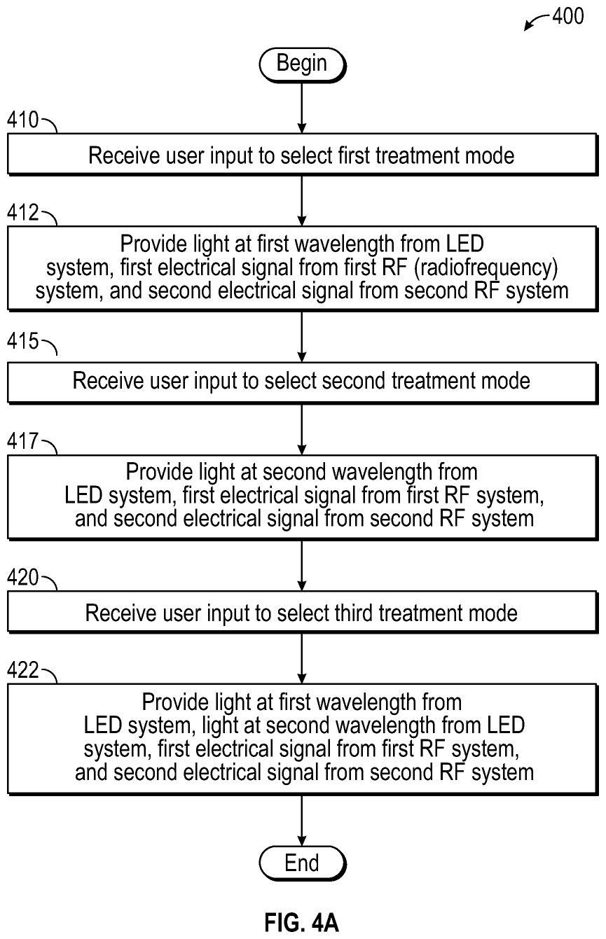

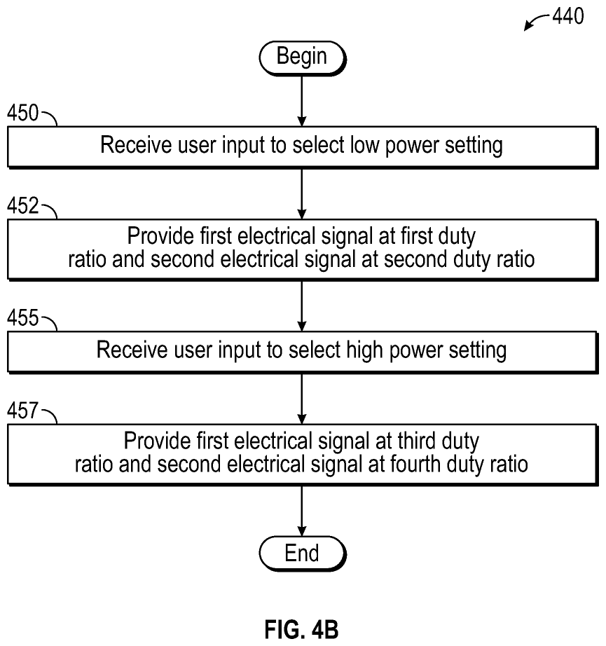

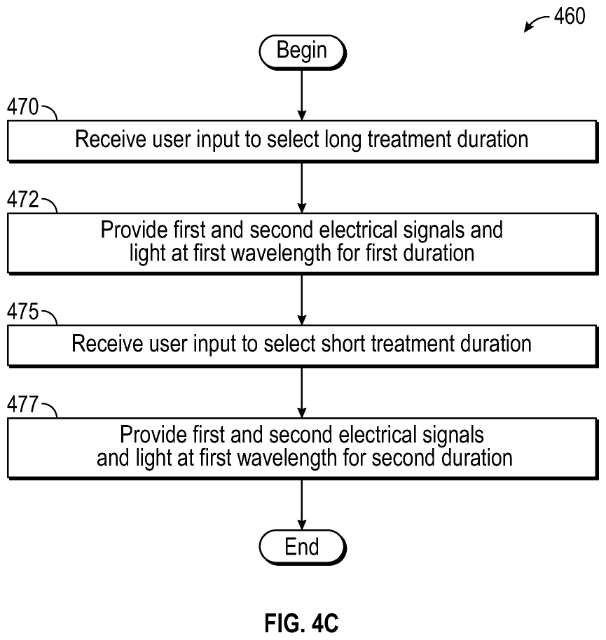

FIG. 4A shows a process 400 for selecting treatment mode performed by a multi-modal skin treatment device of some embodiments. The process 400 begins at 410 by receiving a user input to select a first treatment mode. At 412, in response receiving the user input to select the first treatment mode, the process 400 provides light at a first wavelength from an LED system, a first electrical signal from a first RF (radiofrequency) system, and a second electrical signal from a second RF system. At 415, the process 400 receives a user input to select a second treatment mode.

At 417, in response to receiving the user input to select the second treatment mode, the process 400 provides light at a second wavelength from the LED system, the first electrical signal from the first RF system, and the second electrical signal from the second RF system. At 420, the process 400 receives a user input to select a third treatment mode. At 422, in response to receiving the user input to select the third treatment mode, the process 400 provides light at the first wavelength from the LED system, light at the second wavelength from the LED system, the first electrical signal from the first RF system, and the second electrical signal from the second RF system. The process 400 then ends.

FIG. 4B shows a process 440 for selecting a power setting performed by a multi-modal skin treatment device of some embodiments. The process 440 begins at 450 by receiving a user input to select a low power setting. At 452, in response to receiving the user input to select a low power setting, the process 440 provides the first electrical signal at a first duty ratio and the second electrical signal at a second duty ratio.

At 455, the process 440 receives a user input to select a high power setting. At 457, in response to receiving the user input to select the high power setting, the process 440 provides the first electrical signal at a third duty ratio and the second electrical signal at a fourth duty ratio. The third duty ratio is higher than the first duty ratio, and the fourth duty ratio is higher than the second duty ratio. The process 440 then ends.

FIG. 4C shows a process 460 for selecting a treatment duration performed by a multi-modal skin treatment device of some embodiments. The process 460 begins at 470 by receiving a user input to select a long treatment duration. At 472, in response to receiving the user input to select a long treatment duration, the process 460 provides first and second electrical signals and light at the first wavelength for a first duration.

At 475, the process 460 receives a user input to select a short treatment duration.

At 477, in response to receiving the user input to select a short treatment duration, the process 460 provides the first and second electrical signals and light at the first wavelength for the second duration, where the second duration is shorter than the first duration. The process 460 then ends. In other embodiments, the second duration is longer than the first duration, however, and the user inputs configure the device accordingly.

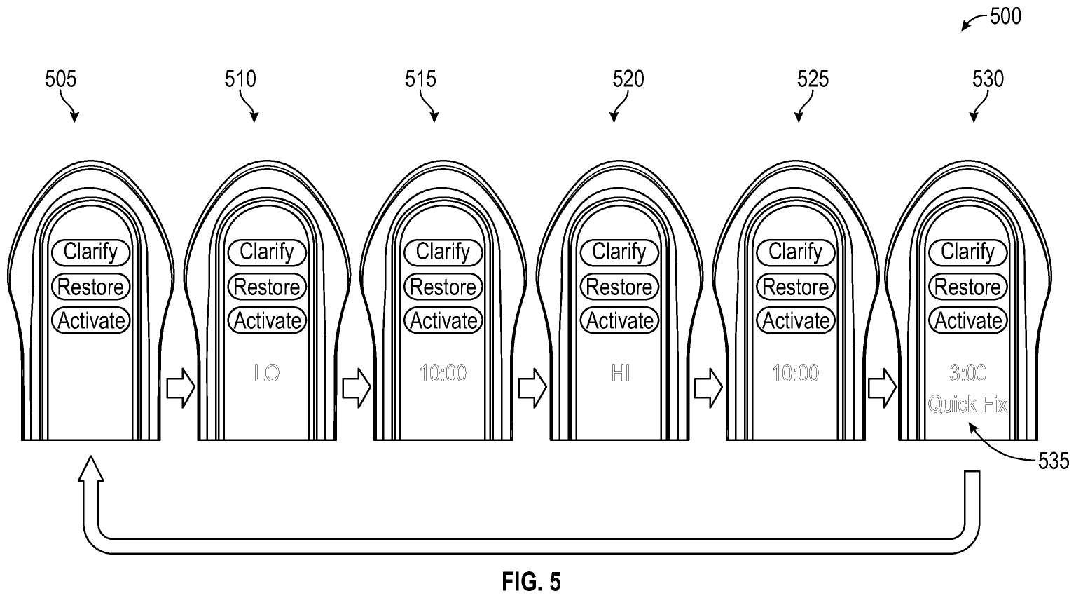

FIG. 5 shows a sequence 500 of user interactions with a user interface to select the treatment mode, power setting, and treatment duration for a multi-modal skin treatment device of some embodiments. In the example shown, in a sequence of user interactions with the user interface of a multi-modal skin treatment device (e.g., device 200 of FIG. 2A) allow a user to select various treatment modes and power settings.

In one example, the device is initially in a powered-off state 505. The user selects the desired treatment mode by pressing, once, upon the corresponding one of the three treatment mode selection buttons 260-262. In this example, the user selects a treatment mode, e.g., the CLARIFY treatment mode, by pressing button 260. The device 200 then enters a first state 510, which corresponds to the low power setting for the treatment mode. In the first state 510, the device 200 indicates to the user that the device is in the treatment mode by illuminating the button 260. The device may also indicate to the user that the device is in the low power setting, by showing text (e.g., “LO”) on the display 265. To further communicate to the user that the device 200 is in the low power setting, the device 200 may also provide an audible sound, such as a beep, at a low volume.

If the user does not further select any buttons, then after a predetermined time (e.g., 2 seconds), the device 200 enters a second state 515, and begins to operate to deliver the low power treatment. The display 265 shows a timer that counts down the remaining duration of the treatment. Since the device 200 is in the low power setting, the timer begins at 10:00 minutes and counts down by seconds until the time runs out and the treatment ends.

If the user selects the button 260 again while the device is in the first state 510 or the second state 515, the device enters a third state 520, which corresponds to the high power setting for the treatment mode (e.g., CLARIFY mode). The device indicates to the user that the device is in the high power setting, by showing text (e.g., “HI”) on the display 265. To further communicate to the user that the device 200 is in the high power setting, the device 200 may also provide an audible sound, such as a beep, at a medium volume, e.g., a volume that is higher than the volume of the beep when entering the first state 510.

If the user does not further select any buttons, then after a predetermined time (e.g., 2 seconds), the device 200 enters a fourth state 525, and begins to operate to deliver the high power treatment. The display 265 shows a timer that counts down the remaining duration of the treatment. Since the device 200 is in the high power setting, the timer begins at 10:00 minutes and counts down by seconds until the time runs out and the treatment ends.

If the user selects the button 260 again while the device is in the third state 520 or the fourth state 525, the device enters a fifth state 530, which corresponds to the quick fix setting for the treatment mode (e.g., CLARIFY mode). The device illuminates a secondary area 535 of the display 265 with text (e.g., “QUICK FIX”). To further communicate to the user that the device 200 is in the quick fix setting, the device 200 may also provide an audible sound, such as a beep, at a high volume, e.g. a volume that is higher than the volume of the beeps when entering the first state 510 or the third state 520. The display 265 also shows a timer that counts down the remaining duration of the treatment. Since the device 200 is in the quick fix setting, the timer begins at 3:00 minutes and counts down by seconds until the time runs out and the treatment ends.

Once the device 200 completes operation in the second state 515, the fourth state 525, or the fifth state 530, or if the button 260 is again pressed by the user during operation in the fifth state 530, then the device returns to the powered-off state 505.

During the sequence 500, the user may at any time select a button corresponding to a different treatment mode, e.g., button 261 or button 262. Doing so may return the device 200 to the powered off state 505, or alternatively may return the device 200 to the first state 510 for the treatment mode corresponding to the button that was pressed.

Though presented for an example treatment mode (e.g., the CLARIFY treatment mode), the discussion regarding FIG. 5 equally applies if the user desires to utilize other treatment modes. Other treatment modes may include a second treatment mode (e.g., the RESTORE treatment mode), a third treatment mode (e.g., the ACTIVATE treatment mode), or other treatment modes. Note that the device 200 depicted in FIG. 5 only shows blue light being provided during the sequence of states 510-530, since the example was for an example treatment mode (e.g., CLARIFY treatment mode). The terms first, second, and third treatment mode are used as examples and do not necessarily correspond to any particular treatment mode described herein or contemplated within the scope of the disclosure.

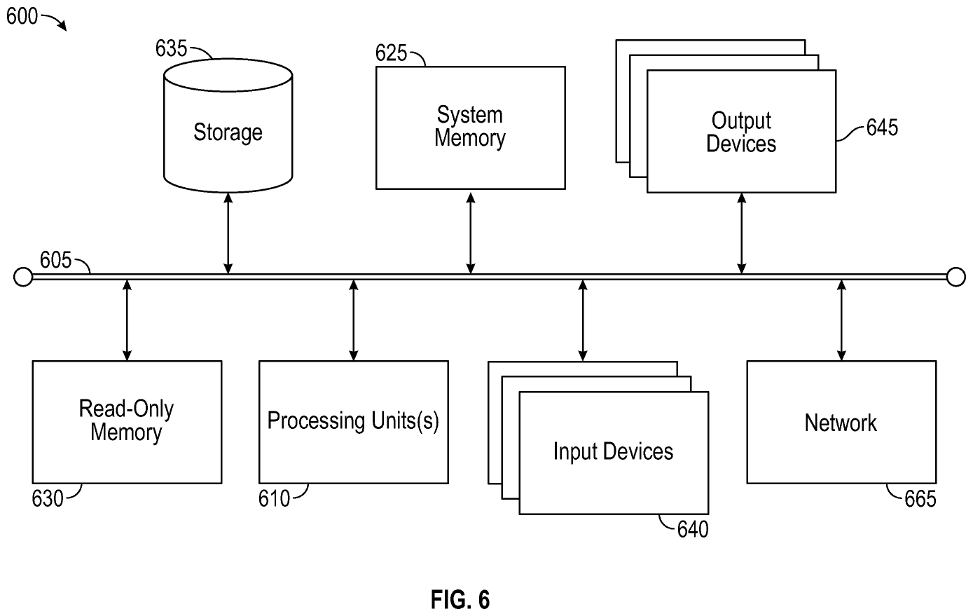

FIG. 6 conceptually illustrates an electronic system 600 with which some embodiments of the invention are implemented. The electronic system 600 can be used to execute any of the control or compiler systems described above in some embodiments. Such an electronic system 600 may include various types of computer readable media and interfaces for various other types of computer readable media. Electronic system 600 includes one or more of a bus 605, processing unit(s) 610, a system memory 625, a read-only memory 630, a permanent storage device 635, input devices 640, and output devices 645.

The bus 605 collectively represents all system, peripheral, and chipset buses that communicatively connect the numerous internal devices of the electronic system 600. For instance, the bus 605 communicatively connects the processing unit(s) 610 with the read-only memory 630, the system memory 625, and the permanent storage device 635.

From these various memory units, the processing unit(s) 610 retrieves instructions to execute and data to process in order to execute the processes of the invention. The processing unit(s) may be a single processor or a multi-core processor in different embodiments.

The read-only-memory 630 (ROM) stores static data and instructions that are needed by the processing unit(s) 610 and other modules of the electronic system. The permanent storage device 635, on the other hand, is a read-and-write memory device. This device is a non-volatile memory unit that stores instructions and data even when the electronic system 600 is off. Some embodiments of the invention use a mass-storage device (such as a magnetic or optical disk and its corresponding disk drive) as the permanent storage device 635.

Other embodiments use a removable storage device (such as a floppy disk, flash drive, etc.) as the permanent storage device. Like the permanent storage device 635, the system memory 625 is a read-and-write memory device. However, unlike permanent storage device 635, the system memory is a volatile read-and-write memory, such a random-access memory. The system memory stores some of the instructions and data that the processor needs at runtime. In some embodiments, the invention's processes are stored in the system memory 625, the permanent storage device 635, or the read-only memory 630. From these various memory units, the processing unit(s) 610 retrieves instructions to execute and data to process in order to execute the processes of some embodiments.

The bus 605 also connects to the input devices 640 and the output devices 645. The input devices enable the user to communicate information and select commands to the electronic system. The input devices 640 may include alphanumeric keyboards and pointing devices (also called “cursor control devices”). The output devices 645 display images generated by the electronic system. The output devices include printers and display devices, such as cathode ray tubes (CRT) or liquid crystal displays (LCD). Some embodiments include devices such as a touchscreen that function as both input and output devices.

The bus 605 also may couple the electronic system 600 to a network 665 through a network adapter (not shown). In this manner, the computer can be a part of a network of computers, such as a local area network (“LAN”), a wide area network (“WAN”), or an Intranet, or a network of networks, such as the Internet. Any or all components of electronic system 600 may be used in conjunction with the invention.

Some embodiments include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, compact discs, digital versatile discs, flash memory, magnetic or solid state hard drives, Blu-Ray® discs, optical discs, floppy discs, or any other optical or magnetic media. The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some embodiments are performed by one or more integrated circuits, such as application specific integrated circuits (ASICs) or field programmable gate arrays (FPGAs). In some embodiments, such integrated circuits execute instructions that are stored on the circuit itself.

As used in this specification, the terms “computer”, “server”, “processor”, and “memory” all refer to electronic or other technological devices. These terms exclude people or groups of people. For the purposes of the specification, the terms display or displaying means displaying on an electronic device. As used in this specification, the terms “computer readable medium,” “computer readable media,” and “machine readable medium” are entirely restricted to tangible, physical objects that store information in a form that is readable by a computer. These terms exclude any wireless signals, wired download signals, and any other ephemeral signals.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the spirit or scope of the disclosure, as appreciated by those skilled in the art in light of the above teachings. It is therefore to be understood that, within the scope of the claims and their equivalents, the invention may be practiced otherwise than as specifically described. Moreover, features described in connection with one embodiment may be used in conjunction with other embodiments, even if not explicitly stated above.

We claim:

1. A device for skin treatment, comprising:
an LED (light-emitting diode) system that is configured to emit light at a first wavelength;
a first radiofrequency (RF) system that is configured to generate a first electrical signal; and
a second RF system that is configured to generate a second electrical signal,
wherein the first RF system, the second RF system, and the LED system are configurable to operate in a first treatment mode, causing the device to provide the first electrical signal, the second electrical signal, and light at the first wavelength.

2. The device of claim 1, wherein the first electrical signal is configured to provide radiofrequency therapy, and the second electrical signal is configured to provide electro-muscular stimulation (EMS) therapy.

3. The device of claim 1,
wherein the LED system is further configured to emit light at a second wavelength,
wherein the first RF system, the second RF system, and the LED system are configurable to operate in a second treatment mode, causing the device to provide the first electrical signal, the second electrical signal, and light at the second wavelength.

4. The device of claim 3,
wherein the first RF system, the second RF system, and the LED system are configurable to operate in a third treatment mode, causing the device to provide the first electrical signal, the second electrical signal, light at the first wavelength, and light at the second wavelength.

5. The device of claim 3, wherein the first wavelength is between 405 and 420 nanometers, and the second wavelength is between 630 and 660 nanometers.

6. The device of claim 1, further comprising:
a first electrode pair operatively connected to the first RF system; and
a second electrode pair operatively connected to the second RF system,
wherein the LED system, the first electrode pair, and the second electrode pair are mounted on a treatment surface at one end of the device.

7. The device of claim 6, wherein the treatment surface has a teardrop shape.

8. The device of claim 1, wherein the device is configurable by a user to operate at one or more of a low power setting and a high power setting.

9. The device of claim 8, wherein the first electrical signal is characterized by a first voltage and the second electrical signal is characterized by a second voltage, wherein at least one of the first voltage and the second voltage are different in the low power setting and the high power setting.

10. The device of claim 8, wherein the first electrical signal is characterized by a first duty cycle and the second electrical signal is characterized by a second duty cycle, wherein at least one of the first duty cycle and the second duty cycle are different in the low power setting and the high power setting.

11. The device of claim 8, wherein the first electrical signal is characterized by a first duty ratio, and the second electrical signal is characterized by a second duty ratio, wherein at least one of the first duty ratio and the second duty ratio are different in the low power setting and the high power setting.

12. The device of claim 1, wherein the device is configurable by a user to operate at one of a long treatment mode and a short treatment mode.

13. The device of claim 12, wherein the device operates for ten minutes in the long treatment mode, and the device operates for three minutes in the short treatment mode.

14. A method for skin treatment, comprising:
receiving a first user input to select a first treatment mode; and
in response to selecting the first treatment mode, providing light at a first wavelength from an LED system, a first electrical signal from a first RF (radiofrequency) system, and a second electrical signal from a second RF system.

15. The device of claim 14, further comprising configuring the first electrical signal to provide radiofrequency therapy.

16. The device of claim 14, further comprising configuring the second electrical signal to provide electro-muscular stimulation (EMS) therapy.

17. The method of claim 14, further comprising:
receiving a second user input to select a second treatment mode; and
in response to selecting the second treatment mode, providing light at a second wavelength from the LED system, the first electrical signal from the first RF system, and the second electrical signal from the second RF system.

18. The method of claim 17, further comprising:
receiving a third user input to select a third treatment mode; and
in response to selecting the third treatment mode, providing light at the first wavelength from the LED system, light at the second wavelength from the LED system, the first electrical signal from the first RF system, and the second electrical signal from the second RF system.

19. The method of claim 14, wherein in response to receiving the first user input, the first electrical signal is provided at a first duty ratio and the second electrical signal is provided at a second duty ratio, the method further comprising:
receiving a second user input to select a high power setting; and
in response to selecting the high power setting, providing the first electrical signal at a third duty ratio,
wherein the third duty ratio is higher than the first duty ratio.

20. The method of claim 19, further comprising, in response to selecting the high power setting, providing the second electrical signal at a fourth duty ratio,
wherein the fourth duty ratio is higher than the second duty ratio.

21. The method of claim 19, wherein in response to receiving one of the first user input and the second user input, the first and second electrical signals and light at the first wavelength are provided for a first duration, the method further comprising:
receiving a third user input to select a short treatment mode; and
in response to selecting the short treatment mode, providing, for a second duration, the first and second electrical signals and light at the first wavelength,
wherein the second duration is shorter than the first duration.
